(19) United States
(12) Patent Application Publication
(10) Pub. No.: US 2024/0136011 A1
(43) Pub. Date: Apr. 25, 2024

(54) SYSTEM, METHOD, AND COMPUTER PROGRAM FOR PHYSICS-BASED BINDING AFFINITY ESTIMATION

(71) Applicant: BOARD OF TRUSTEES OF THE UNIVERSITY OF ARKANSAS, Little Rock, AR (US)

(72) Inventor: Mahmoud Moradi, Fayetteville, AR (US)

(73) Assignee: BOARD OF TRUSTEES OF THE UNIVERSITY OF ARKANSAS, Little Rock, AR (US)

(21) Appl. No.: 18/481,986

(22) Filed: Oct. 5, 2023

Related U.S. Application Data
(60) Provisional application No. 63/379,476, filed on Oct. 14, 2022.

(51) Int. Cl. G16B 15/30; G16B 40/20

(52) U.S. Cl. CPC G16B 15/30; G16B 40/20

(57) ABSTRACT

The invention relates to a system, method, and computer program for estimating binding affinity that is purely physics-based, highly accurate, computationally efficient, and applicable to drugs of any size and proteins of any flexibility level. In one aspect, a method for physics-based binding affinity estimation is disclosed. The method includes the step of creating a unified simulation for a ligand and a target molecule, where the unified simulation includes a plurality of replicas that are each associated with four restraints along four variables. The method also includes the steps of exchanging data for the four variables between a subset of the replicas and performing a single non-parametric reweighting analysis with the data to estimate an absolute binding free energy for the ligand and target molecule.

### CROSS-REFERENCE TO RELATED APPLICATIONS

This application claims the benefit of U.S. Provisional Patent Application No. 63/379,476 filed on Oct. 14, 2022, and incorporates the provisional application by reference in its entirety into this document as if fully set out at this point.

### GOVERNMENT LICENSE RIGHTS

This invention was made with government support under Grant No. CRE1945465 awarded by the National Science Foundation and under Grant No. ACI1548562 awarded by the National Science Foundation. The government has certain rights in the invention.

### BACKGROUND OF THE INVENTION

### 1. Field of the Invention

The subject matter disclosed herein generally relates to a system, method, and computer program for physics-based binding affinity estimation.

### 2. Description of the Related Art

Binding affinity is the strength of the binding interaction between a biomolecule (e.g., protein or DNA) to its ligand/binding partner (e.g., drug or inhibitor). Binding affinity is typically measured and reported by the equilibrium dissociation constant (K_{d}), which evaluates and ranks the order of strengths of biomolecular interactions. The smaller the K_{d} value, the greater the binding affinity of the ligand for its target. The larger the K_{d} value, the weaker the target molecule and ligand are attracted to and bind to one another.

Binding affinity is influenced by non-covalent intermolecular interactions, such as hydrogen bonding, electrostatic interactions, hydrophobic, and Van der Waals forces between the two molecules. In addition, the binding affinity between a ligand and its target molecule may be affected by the presence of other molecules.

Computer modeling can estimate the binding affinity between a ligand and its target molecule, and computational binding affinity estimation is routinely used in computer-aided drug design (CADD). However, accurate quantification of absolute binding affinities remains a problem of significant importance in computational biophysics. In principle, accurate binding free energy calculations should be the cornerstone of any study investigating protein-ligand interactions. However, the high computational costs that typically accompany such calculations necessitate improving the computational methods traditionally used to investigate and analyze complex biomolecular interactions. Experimentally determined binding affinities are commonly used as benchmarks to judge the accuracy of various computational binding affinity estimation methods.

Several experimental techniques can be used to study protein-ligand binding equilibria. For instance, isothermal titration calorimetry (ITC) can detect the interaction of binding partners based on changes in solution heat capacity and binding partner concentration. Other methods such as fluorescence spectroscopy rely on changes in fluorescence intensity upon ligand binding. Surface plasmon resonance (SPR) can calculate binding affinities based on changes in the refractive index when an immobilized binding partner interacts with a free binding partner. Studies have found that experimental binding affinities can vary depending on the experimental method used. Therefore, a thorough understanding of the experimental conditions used to generate reference data is essential when comparing computationally determined binding affinities with experimental values.

Several computational methods at varying levels of rigor and complexity have been used to determine binding affinities for biomolecular interactions. Knowledge-based statistical potentials and force field scoring potentials are typically used to rank docked protein-ligand or protein-protein complexes but can also be used for binding affinity prediction. A significant disadvantage of these methods is that they do not treat the entropic effects rigorously, which effectively decreases the accuracy of such binding affinity predictions. This is also the case for methods like Molecular Mechanics/Poisson-Boltzmann-Surface Area (MM-PBSA) and Molecular Mechanics/Generalized Born-Surface Area (MM-GB SA), which combine sampling of conformations from explicit solvent molecular dynamics (MD) simulations with free energy estimation based on implicit continuum solvent models. Adequate sampling of ligand conformational dynamics and ligand roto-translational movements with respect to the protein is essential for accurately quantifying the entropic reduction arising from the binding event. MM-PBSA/GBSA methods typically neglect the contribution of these entropic terms to the binding free energy.

One of the best-known binding free energy estimation methods is alchemical free energy perturbation (FEP), where scaling of non-bonded interactions enables reversible decoupling of the ligand from its environment in the bound state as well as the unbound state. Most entropic and enthalpic contributors to changes in binding affinity are typically considered during FEP simulations, thus avoiding the approximations used by methods like MM-PBSA/GBSA. A disadvantage of FEP is that ligands tend to move away from the binding site during the decoupling process, resulting in poorly defined target states of the FEP calculation being used as starting conditions for the re-coupling process. Using receptor-ligand restraints to resolve this issue introduces some ambiguity to the way a standard state is defined, with a level of correlation between the size of the simulation cell and the standard state. This can be corrected via the use of appropriate geometrical restraints.

Unrestrained long timescale MD simulations should theoretically allow the investigation and accurate quantification of protein-ligand or protein-protein binding events. While microsecond-level MD simulations provide a more accurate description of protein conformational dynamics as compared to shorter simulations, efficient sampling of the conformational landscape remains a major issue and requires access to timescales beyond the capabilities of current MD simulations. Several methods have been developed to tackle the sampling problem. Markov state models enable the sampling and characterization of native as well as alternative binding states. Similarly, weighted ensemble (WE) simulations sample the conformational landscape along one or more discretized reaction coordinates based on the assignment of a statistical weight to each simulation. More traditionally, umbrella sampling (US) along such reaction coordinates can be used to guide the binding or unbinding of a ligand, after which algorithms like the weighted histogram analysis method (WHAM) can be used to calculate a unidimensional potential of mean force (PMF), which quantifies ligand binding and unbinding along with a reaction coordinate. Better convergence of the calculated free energy profiles can be achieved by exchanging conformations between successive umbrella-sampling windows, such as occurs in bias-exchange umbrella sampling (BEUS). Other methods based on similar principles include umbrella integration, well-tempered metadynamics, adaptive biasing force (ABF) simulations, and variations of these techniques.

Incomplete sampling of important degrees of freedom, such as the orientation of the ligand with respect to the protein, remains a significant disadvantage of unidimensional PMF-based methods. To resolve this problem, a method was devised wherein explicitly defined geometrical restraints on the orientation and conformation of the binding partners are used to reduce the conformational entropy of the biomolecular system being studied. This results in improved convergence of the PMF calculation. The introduction of a restraining potential based on the root-mean-square deviation (RMSD) of the ligand relative to its average bound conformation reduces the flexibility of the ligand and the number of conformations that need to be sampled. This method avoids decoupling the ligand from its surrounding environment as required by alchemical FEP.

### SUMMARY OF THE INVENTION

The invention relates to a system, method, and computer program for physics-based binding affinity estimation that employs state-of-the-art free energy calculation methods based on all-atom molecular dynamics simulations. The inventive system, method, and computer program can estimate the binding affinity of a drug to its target molecule with higher accuracy than existing techniques. While the current methods and CADD software provide affinity estimates, these estimates are generally rough docking scores rather than true binding affinities that heavily rely on empirically fitted formulas rather than solely physics-based methods. Enhanced sampling molecular dynamics (MD) methods such as umbrella sampling (US) provide the purest physics-based approach to absolute binding free energy calculation that can, in principle, accurately estimate the binding affinity within the approximations imposed by empirical force fields.

However, inadequate sampling of slow degrees of freedom, which results in the so-called quasi-nonergodicity, has been an important practical limitation of these methods. Restraining slow degrees of freedom provides a potential solution to this problem. The restraining approach requires follow-up potential of mean force (PMF) calculations along the restrained degrees of freedom. Various restraining protocols have been proposed within the context of both alchemical and geometric enhanced sampling methods. An important problem that has discouraged the use of this scheme is the need to set up numerous enhanced sampling simulations and to estimate various free energy and correction terms based on these independent simulations.

Accordingly, it is an object of this invention to provide an improved system, method, and computer program for estimating binding affinity that is purely physics-based, highly accurate, computationally efficient, and applicable to drugs of any size and proteins of any flexibility level.

Another object of this invention is to provide a system, method, and computer program for physics-based binding affinity estimation that is an alternative to the existing methods for binding affinity estimation, which: (1) like the FEP method and the US method is purely physics-based; but (2) unlike the FEP method applies to larger drugs and flexible proteins and drugs; and (3) unlike the US method is accurate and reliable without needing to be corrected with complicated and computationally expensive methods.

Another object of this invention is to provide a system, method, and computer program for physics-based binding affinity estimation based on a bias-exchange restrained (BER) MD simulation technique that requires setting up only one multi-copy simulation and performing one non-parametric reweighting (NPR) analysis to accurately estimate the absolute binding free energy without the need to perform and analyze various simulations. The invention disclosed herein provides a seamless setup and analysis and superior accuracy, which is due to the lack of discontinuity in the sampled configurational space.

A further object of this invention is to provide a system, method, and computer program for estimating binding affinity that can be used to provide absolute drug binding affinities rather than relative binding affinities compared to its existing alternatives.

In general, in a first aspect, the invention relates to a system that includes a computer, where the computer has a processor and a memory, and a software module stored in the memory. The software module includes executable instructions that, when executed by the processor, cause the processor to generate a model for a ligand bound to a target molecule and estimate a binding affinity between the ligand and the target molecule. Binding affinity is estimated by creating a unified simulation with a plurality of replicas, where each replica is associated with four restraints along four variables, and exchanging data for the four variables between a subset of the replicas. A single non-parametric reweighting analysis is then performed on the data to estimate an absolute binding free energy for the ligand and the target molecule.

In one embodiment, the processor is configured to generate the model using at least one docking method.

In one embodiment, the processor is configured to generate the model using x-ray crystallography.

In one embodiment, the processor is configured to generate the model by simulating the ligand and the target molecule in a box of water and ions.

In one embodiment, the four variables include a distance between the mass centers of the ligand and the target molecule (d), an orientation of the ligand (Q), a root-mean-square-deviation of the ligand with respect to a reference structure (r_{L}), and a root-mean-square-deviation of the target molecule with respect to the reference structure (rₚ).

In one embodiment, the ligand is a drug, and the target molecule is a protein.

In general, in a second aspect, the invention relates to a method for physics-based binding affinity estimation. The method includes the step of creating a unified simulation for a ligand and a target molecule, where the unified simulation includes a plurality of replicas, each replica associated with four restraints along four variables. The method also includes the steps of exchanging data for the four variables between a subset of the replicas and performing a single non-parametric reweighting analysis with the data to estimate an absolute binding free energy for the ligand and the target molecule.

In one embodiment, the four variables include a distance between the mass centers of the ligand and the target molecule (d), an orientation of the ligand (Ω), a root-mean-square-deviation of the ligand with respect to a reference structure (r_{L}), and a root-mean-square-deviation of the target molecule with respect to the reference structure (rₚ).

In one embodiment, the step of creating the unified simulation involves simulating varying distances between the ligand and the target molecule. The distance between the ligand and the target molecule in a first replica reflects the ligand and the target molecule when completely bound. The distance between the ligand and the target molecule in a second replica reflects the ligand and the target molecule when completely unbound. The distances between the ligand and the target molecule in the remaining replicas falls between those of the first replica and the second replica.

In one embodiment, the step of simulating varying distances between the ligand and the target molecule includes performing all the simulations simultaneously.

In one embodiment, the step of exchanging data for the four variables includes the steps of establishing exchange rules to connect the plurality of replicas, where each replica is associated with one of a plurality of nodes in the unified simulation, and using the replicas to move between the nodes.

In one embodiment, the replicas have different centers for the four restraints.

In one embodiment, the step of performing the single non-parametric reweighting analysis includes the steps of estimating a likelihood of finding the ligand in the bulk versus finding the ligand at a specific orientation and conformation within a binding pocket of the target molecule; estimating a difference between flexibility for the ligand in the bulk versus within the binding pocket in terms of movement; estimating a difference between flexibility for the ligand in the bulk versus within the binding pocket in terms of fluctuations in root-mean-square deviation; and estimating a difference between flexibility for the ligand in the bulk versus within said binding pocket in terms of orientational changes. In one embodiment, the step of performing the single non-parametric reweighting analysis includes the step of estimating ΔG⁰ according to the equation

$$Δ {G}^{0} = - RT ln \frac{{\sum }_{i} {w}_{i} {𝒳}_{i}^{pocket}}{{\sum }_{i} {w}_{i} {𝒳}_{i}^{bulk} {p}_{i}} .$$

In one embodiment, the method further includes the step of recording the data exchanged between the subset of replicas.

In general, in a third aspect, the invention relates to a method for physics-based binding affinity estimation. The method includes the steps of generating a model for a ligand bound to a target molecule and creating a unified simulation for the ligand and the target molecule, where the unified simulation includes a plurality of replicas, each replica associated with four restrains along four variables. The method also includes the steps of exchanging data for the four variables between a subset of the replicas, recording the data, and performing a single non-parametric reweighting analysis with said data to estimate an absolute binding free energy for the ligand and the target molecule.

In one embodiment, the four variables include a distance between the mass centers of the ligand and the target molecule (d), an orientation of the ligand (Q), a root-mean-square-deviation of the ligand with respect to a reference structure (r_{L}), and a root-mean-square-deviation of the target molecule with respect to the reference structure (rₚ).

In one embodiment, the step of exchanging data for the four variables includes the steps of establishing exchange rules to connect the plurality of replicas, where each replica is associated with one of a plurality of nodes in the unified simulation, and using the replicas to move between the nodes.

In one embodiment, the step of performing the single non-parametric reweighting analysis includes the steps of estimating a likelihood of finding the ligand in the bulk versus finding the ligand at a specific orientation and conformation within a binding pocket of the target molecule; estimating a difference between flexibility for the ligand in the bulk versus within the binding pocket in terms of movement; estimating a difference between flexibility for the ligand in the bulk versus within the binding pocket in terms of fluctuations in root-mean-square deviation; and estimating a difference between flexibility for the ligand in the bulk versus within said binding pocket in terms of orientational changes.

### BRIEF DESCRIPTION OF THE DRAWINGS

The above and other objects and advantages of this invention may be more clearly seen when viewed in conjunction with the accompanying drawing wherein:

FIG. 1 is a schematic representation of a prior stratification strategy for absolute binding free energy calculations using 1D BEUS simulations along four different collective variables including d, Ω, r_{L}, and rₚ. Each dashed box represents an independent 1D BEUS simulation and each solid box represents a single replica of the system. The conditions imposed on each BEUS simulation is also provided (e.g., “no ligand” indicating that the system is an apo protein or “Ω=0” indicating a restraint on Ω at 0). The window center for each simulation is provided in solid boxes. The center of d may be d₀ (representing the binding pocket), d₁, d₂, . . . , or d_{N} (representing the bulk), while the center of Ω may be 0, 1, . . . , or N_{Ω}, where its unit determines the window size. Similarly, for rₚ and r_{L}, centers could vary from 0 to Nₚ and N_{L}, respectively, where the units determine the window size. The arrows represent the possible exchanges within a 1D BEUS scheme. Black, red, blue, and green arrows represent exchanging d, Ω, rₚ, and r_{L} biases, respectively.

FIG. 2 is a schematic representation of an example of the inventive system, method, and computer program for absolute binding free energy calculations. Each solid box represents a replica of the system that is associated with four restraints along four collective variables including d, Ω, r_{L}, and rₚ. For more details on window centers see FIG. 1. The dashed box represents the part of the graph that is associated with the translational separation of the ligand from the protein and is equivalent to the black dashed box in FIG. 1; however, all systems in FIG. 2 are either directly or indirectly connected forming a connected graph, allowing a system to move from any point in the sampled space to any other point, given enough time.

FIGS. 3A and 3B graphically illustrate binding free energy measurements from FEP simulations with the forward and backward transformations shown in red and blue respectively, where free energy change for the double annihilation of heparin hexasaccharide is shown in its bound state (3A) and in its free state (3B)—short FEP, and in its bound state (3C) and in its free state (3D)—long FEP.

FIG. 4 is a flowchart representing an example of the inventive system, method, and computer program for absolute binding free energy calculations.

FIG. 5 is a schematic representation of an example of the inventive system, method, and computer program for absolute binding free energy calculations.

### DETAILED DESCRIPTION OF THE INVENTION

While this invention is susceptible to embodiment in many different forms, there are shown in the drawings and will herein be described hereinafter in detail some specific embodiments of the invention. It should be understood, however, that the present disclosure is to be considered an exemplification of the principles of the invention and is not intended to limit the invention to the specific embodiments so described.

The invention relates to a system, method, and computer program for physics-based binding affinity estimation. The inventive physics-based binding affinity estimation method estimates the relative likelihood of finding a drug associated with the protein (bound) compared to being free in the bulk solution (unbound) without using any empirical data to estimate the binding affinity or its correction. The inventive physics-based binding affinity estimation provided herein does not require performing multiple independent simulations and analyses, and the unified simulation setup of the inventive system, method, and computer program primarily prevents discontinuity in the sampled configurational space, which is the manifestation of quasi-nonergodicity problem in independent biased simulations.

Binding affinity is often quantified using the equilibrium dissociation constant (K_{d}), defined as:

K_{d}=[P][L]/[P; L] (Equation 1)

where [P], [L], and [P:L] are the concentrations of protein, ligand, and the protein-ligand complex, respectively. Computationally, the absolute binding free energy (ΔG°), which is the standard molar free energy of binding, is more convenient to calculate. The dissociation constant and the absolute binding free energy are related via

$$Δ G ° = RT ln \frac{{K}_{d}}{1 M} \qquad ( Equation 2 )$$

where R is the gas constant and T is the temperature. Various strategies have been used to estimate ΔG°. Absolute binding free energy or ΔG° is the free energy change associated with moving the ligand from the bulk to the binding pocket. Within the formalism presented in this work, ΔG° is determined from the grid PMF G(x), where x is the position of the ligand mass center from the center of the binding pocket, G(x) is the potential of mean force (PMF) associated with the ligand position x. In practice, we need to bin the 3D space and define the PMF at every bin or grid point as:

G(x)=−RT In p(x) (Equation 3)

where p(x) is the probability of finding the ligand at bin x.

The strategies then define ΔG(x)=G(x)−G(0), where x=0 (i.e., the center of the binding pocket) is defined as the grid point associated with the lowest grid PMF. Recent strategies show that:

ΔG°=−αG(x_{B})+ΔG_{V} (Equation 4)

where x_{B} is any grid point in the bulk, ΔG(x_{B}) is the PMF difference between the binding pocket center and the bulk, and ΔG_{V} is the binding free energy contribution of the volume difference between the binding pocket and the bulk:

$$Δ {G}_{V} = - RT ln \frac{{V}_{P}}{{V}_{B}} \qquad ( Equation 5 )$$

where V_{B}≈1661 Å³ is the bulk volume per protein associated with the standard

concentration of 1 M and Vₚ is the binding pocket volume defined as:

$${V}_{P} = {\int }_{pocket} {e}^{- \frac{Δ G ( x )}{RT}} dV \qquad ( Equation 6 )$$

in which the binding “pocket” refers to all x where the ligand is considered bound.

Determining both ΔG(x_{B}) and Vₚ requires finding the grid PMF ΔG(x). However, the scheme finds ΔG(x) within the binding pocket, which is typically a limited space, and in the bulk, which is large but assumed to be completely uniform. More specifically, the scheme determines ΔG(x) in the binding pocket relative to the bulk. This typically requires a transformation from the binding pocket to the bulk and/or vice versa, whether a geometric route is used or an alchemical one.

ΔG(x) of the binding pocket relative to the bulk can be determined by pulling the ligand out of the binding pocket towards the bulk and using an enhanced sampling technique such as US to sample the distance between the mass centers of the ligand and protein (d). ΔG(x) can be estimated, in principle, for all sampled grid points x using this distance-based US simulation.

Note that the collective variable used for biasing would be d, while one may use a different collective variable for PMF calculations, such as x, using an NPR algorithm. One may thus estimate the grid PMF from the distance-based US simulations. ΔG (x) can also be used to estimate Vₚ as defined in Equation 6. There is often no need to strictly define the binding pocket since only low ΔG(x) values have nonnegligible contribution to Vₚ and thus even if included with all sampled grid points, only those close to the binding pocket center have nonnegligible contributions.

As discussed previously above, a main problem with both geometric and alchemical transformation techniques is the quasi-nonergodicity problem due to insufficient sampling of the slow degrees of freedom such as ligand orientation. A recently proposed strategy based on restrained US (RUS) technique has some similarities and some important differences with the inventive method disclosed herein. Therefore, below first briefly describes the RUS scheme for absolute binding free energy calculations, followed by the BER physics-based binding affinity estimation method disclosed herein.

In the RUS scheme, slow degrees of freedom are restrained in a distance-based US simulation and follow-up PMF calculations are used to correct the binding free energy according to:

$$Δ G ° = - Δ {G}_{{r}_{L} , {r}_{P} , Ω} ( {x}_{B} ) + Δ {U}_{Ω} ( {x}_{B} ) + Δ {U}_{{r}_{P}}^{Ω} ( {x}_{B} ) + Δ {U}_{{r}_{L}}^{{r}_{P} , Ω} ( {x}_{B} ) + Δ {G}_{V} \qquad ( Equation 7 )$$

where ΔGᵣ_{_{L}}_{,r}_{ₚ}_{,Ω}(x_{B}) is the same as ΔG(x) except for the presence of harmonic restraints on collective variables: (1) orientation of the ligand (Ω), (2) root-mean-square-deviation (RMSD) of the ligand with respect to a reference structure (r_{L}), and (3) RMSD of the protein with respect to a reference structure (rₚ). The correction term ΔU_{Ω}(x_{B})=U_{Ω}(x_{B})−U_{Ω}(x₀) is defined such that:

$${U}_{Ω} ( x ) = - RT ln \frac{{\int }_{0}^{π} {e}^{- \frac{F ( Ω , x ) + \frac{1}{2} {k}_{Ω} {Ω}^{2}}{RT}} d Ω}{{\int }_{0}^{π} {e}^{- \frac{F ( Ω , x )}{RT}} d Ω} \qquad ( Equation 8 )$$

in which F(Ω, x) is the PMF along Ω while the ligand is at x and k_{Ω} is the force

constant used for restraining Ω. Similarly, ΔUᵣ_{ₚ}^{Ω}(x_{B}) is defined such that:

$${U}_{{r}_{P}}^{Ω} ( x ) = - RT ln \frac{{\int }_{0}^{∞} {e}^{- \frac{F ( {r}_{P} , Ω = 0 , x ) + \frac{1}{2} {k}_{P} {r}_{P}^{2}}{RT}} {dr}_{P}}{{\int }_{0}^{∞} {e}^{- \frac{F ( {r}_{P} , Ω = 0 , x )}{RT}} {dr}_{P}} \qquad ( Equation 9 )$$

in which F(rₚ,Ω=0, x) is the PMF along rₚ while the ligand is at x and while the Ω is restrained at 0, and kₚ is the force constant used for restraining rₚ. Finally, ΔUᵣ_{_{L}}^{r}^{ₚ}^{, Ω}(x_{B}) is defined similarly based on F(r_{L}, rₚ=0, Ω=0, x), which is the PMF along r_{L} while the ligand is at x and while the Ω and rₚ are both restrained at 0 and k_{L} is the force constant used for restraining r_{L}:

$${U}_{{r}_{L}}^{Ω , {r}_{P}} ( x ) = - RT ln \frac{{\int }_{0}^{∞} {e}^{- \frac{F ( {r}_{L} , {r}_{P} = 0 , Ω = 0 , x ) + \frac{1}{2} {k}_{L} {r}_{L}^{2}}{RT}} {dr}_{L}}{{\int }_{0}^{∞} {e}^{- \frac{F ( {r}_{L} , {r}_{P} = 0 , Ω = 0 , x )}{RT}} {dr}_{L}} \qquad ( Equation 10 )$$

Finally, given that ΔG(x)≈ΔGᵣ_{_{L}}_{, r}_{ₚ}_{,Ω}(x) for x in the binding pocket:

$$Δ {G}_{V} \approx - RT ln {\int }_{pocket} {e}^{- \frac{Δ {G}_{{r}_{L} , {r}_{P} , Ω} ( x )}{RT}} \frac{dV}{{V}_{B}} \qquad ( Equation 11 )$$

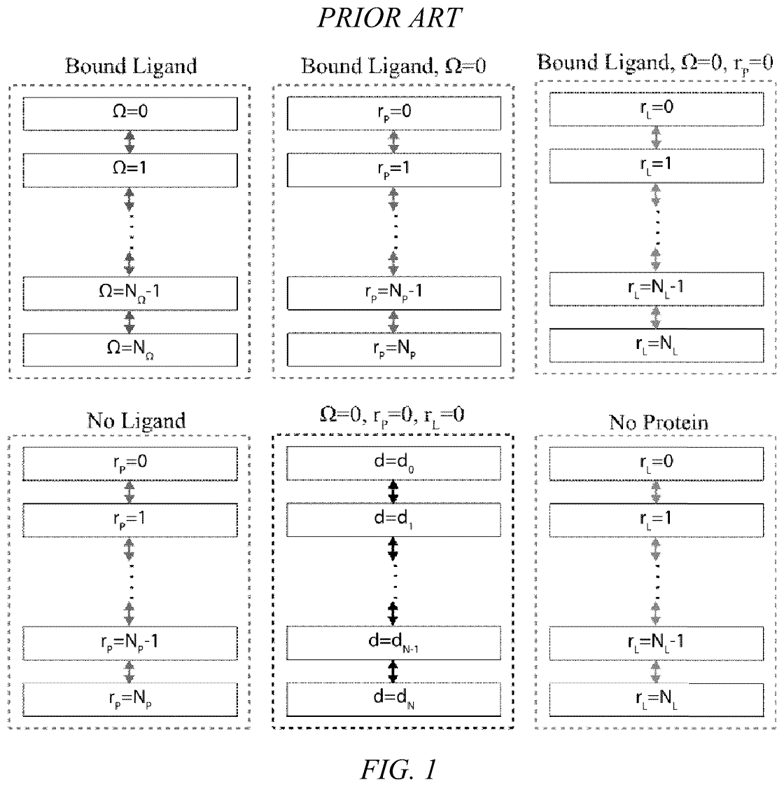

FIG. 1 schematically illustrates the simulations needed to estimate ΔGᵣ_{_{L}}_{, r}_{ₚ}_{,Ω}(x_{B}) and ΔGᵥ(black dashed box in FIG. 1) as well as the PMFs that are needed to estimate ΔU_{Ω}(x_{B}) (i.e., F(Ω|x₀) determined from red dashed box in FIG. 1, ΔUᵣ_{ₚ}^{Ω(x}_{B}) (i.e., F(rₚ, Ω=0, x₀) and F(rₚ, Ω=0, x_{B}) determined from the green dashed boxes in FIG. 1, and ΔUᵣ_{_{L}}^{r}^{ₚ}^{, Ω}(x_{B}) (i.e., F(r_{L}, rₚ=0, Ω=0, x₀) and F(r_{L}, rₚ=0, Ω=0, x_{B}) determined from blue dashed boxes in FIG. 1. Note that F(Ω, x_{B}) is determined analytically.

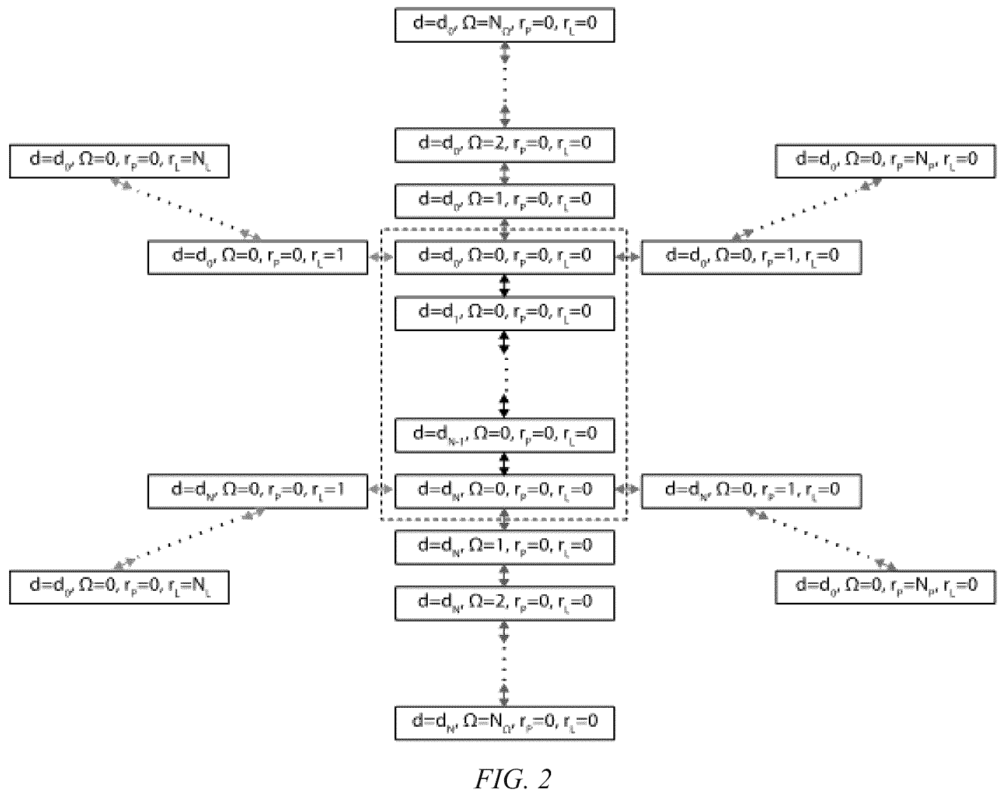

The above methodology in connection with FIG. 1 has proven to be efficient in estimating absolute binding free energies; however, it requires performing six (6) independent US simulations, followed by six (6) independent NPR analyses. The inventive methodology relies on only one (1) set of simulations rather than six (6) independent RUS simulations and even more importantly relies on a single NPR analysis rather than six (6) independent analyses. The above methodology can be performed using either conventional US or BEUS with restraints (RUS or RBEUS) while the inventive method disclosed herein requires a bias-exchange scheme within the BER scheme. BER is more general than both RUS and RBEUS and allows for enhanced sampling along arbitrary connected graphs within a multi-dimensional collective variable space. The BER scheme allows for sampling specific pre-assigned regions of the configuration space, a feature which is essential for the proposed absolute binding free energy calculation method. The inventive method is a specific BER based scheme to sample the most important regions of the configuration space for the specific purpose of absolute binding free energy calculations. As shown in FIG. 2, the BER simulations are done in the 4-dimensional collective variable space of (d, Ω, r_{L}, rₚ). While sampling the entire 4-dimensional space is not feasible, sampling a small portion of this space as specified in FIG. 2 provides the most necessary piece of data for absolute binding free energy estimations. While the two methods have some similarities particularly in the choice of collective variables, both the sampling protocol and the analysis are substantially different.

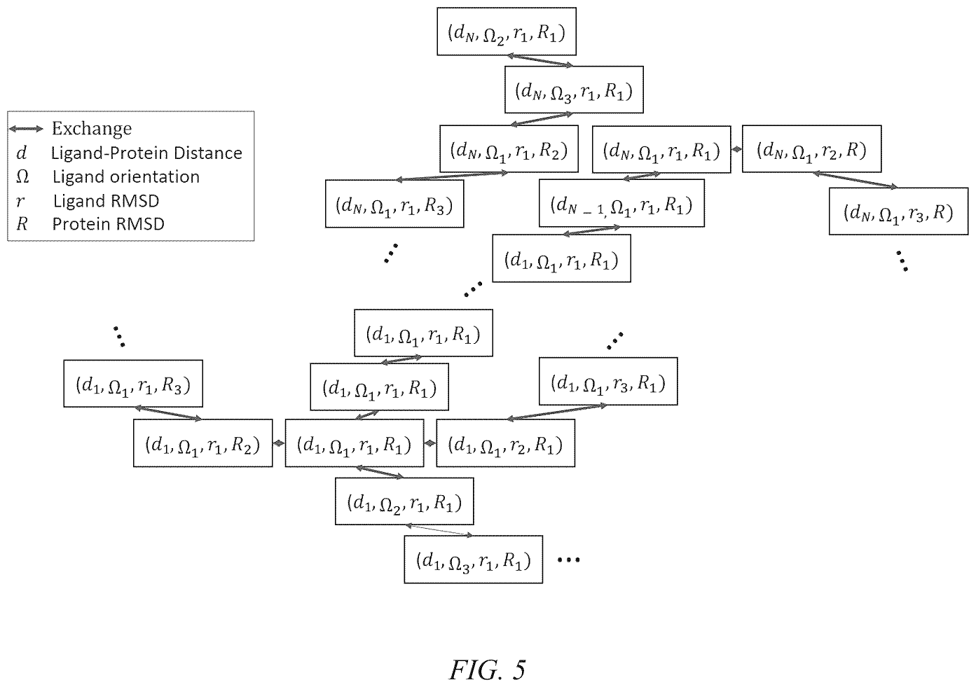

In the inventive BER method, every replica of the system has exactly four (4) restraints—this is not the case in the RUS based BAE method. The difference between different replicas is the centers of the restraints. The exchange rules are defined similar to other bias-exchange methods based on the Hamiltonian and a Metropolis criterion that preserves the details balance. The exchanging replicas are illustrated in FIGS. 2 and 5. The exchange rules “connect” different “nodes” of the graph such that the inventive BER method can be thought of as sampling over a connected graph. Since exchanging replicas allow for a replica to jump from one window/node to another, in principle it is possible for a replica to start from any window/node in the graph and move to any other window/node after a number of stochastically observed jumps. This allows for a continuous sampling of the configuration space, which is missing in the other BAE algorithms including the RUS or RBEUS method.

The analysis of the inventive BER method is exemplified below. A single NPR method can be used to estimate both ΔG⁰ using ΔG°=G_{pocket}−G_{bulk}, where:

$${G}_{pocket} = - RT ln {\int }_{pocket} {\int }_{0}^{π} {\int }_{0}^{∞} {\int }_{0}^{∞} {e}^{- \frac{F ( {r}_{L} , {r}_{P} , Ω , x )}{RT}} {d}_{{r}_{L}} {d}_{{r}_{P}} d Ω dV \qquad ( Equation 12 )$$

$${G}_{bulk} = - RT ln {V}_{B} {\int }_{0}^{π} {\int }_{0}^{∞} {\int }_{0}^{∞} {e}^{- \frac{F ( {r}_{L} = ∞ , {r}_{P} , Ω , x )}{RT}} {d}_{{r}_{L}} {d}_{{r}_{P}} d Ω \qquad ( Equation 13 )$$

While Equations 12 and 13 are exact, in practice, we use approximating expressions:

$$Δ {G}^{0} = - RT ln \frac{{\sum }_{i} {w}_{i} {𝒳}_{i}^{pocket}}{{\sum }_{i} {w}_{i} {𝒳}_{i}^{bulk} {p}_{i}} \qquad ( Equation 14 )$$

The summations go over all sampled configurations in the BER simulation, where i is an index referring to one such sample. wᵢ is the weight associated with the sample determined based on the NPR algorithm (discussed further in the Examples). One may use collective variable d to approximately determine Xᵢ ^{pocket}, and Xᵢ^{pocket} is 1 (0) if in sample i the ligand is (not) in the binding pocket. Xᵢ^{bulk} is 1 (0) if in sample i the ligand is (not) in the bulk. One may only choose highly sampled grid points to represent the bulk and ignore the samples associated with undersampled grid points even if they are considered to be in the bulk. pᵢ is the contribution of each grid point calculated as

$$\frac{{V}_{B}}{{V}_{S}} \frac{{Ω}_{free}}{{Ω}_{i}} ,$$

where V_{B}≈1661 Å³ (the theoretical volume of the bulk), V_{S} is the volume of the grid points used here to represent the bulk Ω_{free} is the effective orientational volume of a free rigid body, and Ωᵢ is the effective orientational volume of the ligand in the grid point associated sample i.

### EXAMPLES

The system, method, and computer program for physics-based binding affinity estimation are further illustrated by the following examples, which are provided for the purpose of demonstration rather than limitation. In the following examples, the inventive system, method, and computer program were used to estimate the binding affinity of human fibroblast growth factor 1 (hFGF1) with heparin hexasaccharide, its glycosaminoglycan (GAG) binding partner.

hFGF1 is an important signaling protein that is implicated in physiological processes, such as cell proliferation and differentiation, neurogenesis, wound healing, tumor growth, and angiogenesis. GAGs are linear anionic polysaccharides that interact with positively charged regions of fibroblast growth factor binding partners to regulate their biological activity. The hFGF1-heparin complex is the most well-known and broadly characterized protein-GAG complex. Heparin binding is thought to stabilize hFGF1 and impart protection against proteolytic degradation.

In the following examples, the inventive system, method, and computer program demonstrate the absolute binding affinity for the hFGF1-heparin interaction in agreement with binding affinity data from isothermal calorimetry (ITC) experiments. The results are also compared with previous methodologies and FEP to show the superiority of the inventive physics-based binding affinity estimation method.

### MD Simulation Details.

The simulations were based on the x-ray crystal structure of the dimeric complex with a heparin hexasaccharide (PDB:2AXM, resolution: 3.0 angstroms). One of the hFGF1 protomers was removed leaving one protein and one ligand in the model of the holo protein. The heparin hexasaccharide consists of N, O6 disulfo-glucosamine and 2-O-sulfo-alpha-L-idopyranuronic acid repeats. The models were solvated in a box of TIP3P waters and 0.15 M NaCl. MD simulations were performed using the NAMD 2.13 simulation package with the CHARMM36m all-atom additive force field. Initially, the systems were energy-minimized for 10,000 steps using the conjugate gradient algorithm. Subsequently, the systems were relaxed using restrained MD simulations in a stepwise manner using the standard CHARMM-GUI protocol. The initial relaxations were performed in an NVT ensemble while the production runs were performed in an NPT ensemble. Simulations were carried out using a 2-fs time step at 300 K using a Langevin integrator with a damping coefficient of γ=0.5 ps⁻¹. The pressure was maintained at 1 atm using the Nosé-Hoover Langevin piston method. The smoothed cutoff distance for non-bonded interactions was set to 10-12 Å and long-range electrostatic interactions were computed with the particle mesh Ewald (PME) method. The initial equilibration lasted 15 nanoseconds.

### Steered Molecular Dynamics (SMD) simulations.

The final conformations of the hFGF1-heparin were used to generate starting conformations for the non-equilibrium pulling simulations. Four (4) collective variables were used for the SMD simulations: (1) distance between the heavy-atom center of mass of heparin and that of the protein (d), (2) the orientation angle of heparin with respect to the protein (Ω), (3) RMSD of the protein (rₚ), and (4) RMSD of heparin (r^{L}). Seven (7) sets of SMD simulations were performed, each for 10 ns. The distance-based SMD simulation was used to pull the heparin away from the protein (10 Å→40 Å), while the other collective variables were restrained. Similarly, the SMD was used to pull Ω from 0°→72° while the rₚ and r_{L} were pulled from 0 Å→3.5 Å and 0 Å→5.5 Å, respectively, while the other collective variables are restrained. The force constant was 100 kcal/(mol.Å²) for the distance and RMSD based restraints and 100 kcal/(mol.degree²) for the orientation-based restraint.

### Bias Exchange Restrained (BER) MD Simulations

Bias exchange restrained MD or BER is a generalization of the bias-exchange umbrella sampling (BEUS), which in turn is a variation of the US simulation method. FIG. 2 illustrates the BER setup used in these examples. Four (4) restraints were used on d, Ω, rₚ, and r_{L} with Δd=1 Å, ΔΩ=3°, and Δrₚ=Δr_{L}=0.5 Å. The force constants used were k_{d}=2 kcal/(mol.Δ²), k_{Ω}=0.5 kcal/(mol.degree²), and kₚ=k_{L}=1 kcal/(mol.Å²). The examples also use the same initial and final values for the four collective variables as in the SMD simulations above. The simulation time was 7 ns including 2 ns of equilibration and 5 ns of data collection.

### Non-parametric reweighting (NPR).

Once the BEUS simulations described above were converged, a non-parametric reweighting method, which is somewhat similar to the multi-state Bennett acceptance ratio method, was used to construct the PMF. The grid PMF and its various estimates provide a simple conceptual framework to understand how restraining can be accounted for with appropriate correction terms. The average grid PMF in terms of the ligand-protein distance provides an alternative to the PMF in terms of d as is often constructed. The non-parametric reweighting allows for calculating the grid PMF in terms of the distance from the center of the binding pocket, eliminating the need for calculating the PMF in terms of the polar and azimuthal angles as in prior methods.

In this method, each sampled configuration is assigned a weight, which can be used to construct the PMF in terms of a desired collective variable. Suppose that a system is biased (for instance, within a BER or US scheme) using N different biasing potentials Uᵢ(r), where i=1, . . . N, and r represents all atomic coordinates. Typically, Uᵢ(r) is a harmonic potential defined in terms of a collective variable with varying centers for different i. Assuming an equal number of sampled configurations from each of the N generated trajectories, we can combine them in a single set of samples {rₖ} (irrespective of which bias was used to generate each sample rₖ) and determine the weight of each sample as:

wₖ=c/Σᵢe^{-β(U}^{ᵢ}^{(r}^{ₖ}^{)-F}^{ᵢ}⁾ (Equation 15)

where c is the normalization constant such that Σₖwₖ=1 and both {wₖ} and {Fᵢ} are determined iteratively using the above equation and the following:

e^{-βF}^{ᵢ}=Σₖwₖe^{-βU}^{ᵢ}^{(r}^{ₖ}⁾ (Equation 16)

Converged wₖ values can be used to construct any ensemble averages such as those discussed above (Equation 14) or the PMF along a given collective variable.

### Alchemical Free Energy Perturbation (FEP) Simulations

Alchemical FEP simulations were performed to calculate the absolute binding free energy for the interaction of hFGF1 with heparin hexasaccharide as a benchmark. The examples used a double annihilation protocol, wherein the heparin hexasaccharide is annihilated in both the free and bound states. The final conformations of the hFGF1-heparin complex and free heparin hexasaccharide equilibrium simulations (discussed previously) were used to generate starting conformations for the bound hFGF1-heparin and free heparin FEP simulations respectively. Two (2) sets of FEP simulations (short and long) were performed for both the hFGF1-heparin complex and the free heparin. The short FEP simulations were performed bi-directionally using 20 λ-windows. Each λ-window included a 0.5-ns equilibration and 2.5 ns of averaging for both the unbound and bound states, for a total of 240 ns. Similarly, the long FEP simulations were also performed bi-directionally using 50 k-windows. Each k-window included a 0.5-ns equilibration and 5 ns of averaging for both the unbound and bound states, for a total of 1.1 μs. Soft-core potential is used for vdW interactions with radius-shifting coefficient of 5.0. The vdW and electrostatic interactions use a different schedule for changing, where the electrostatic interactions are completely decoupled at λ=0.5. All FEP simulations were performed using the NAMD 2.13 simulation package with the CHARMM36m all-atom additive force field, using the protocol discussed previously for the equilibrium simulations. The ParseFEP plugin in VMD was used to analyze the FEP data. Estimates for the absolute binding free energy calculations were obtained using the Bennett acceptance-ratio (BAR) method.

Based on the foregoing, the absolute binding free energy was calculated for the interaction of hFGF1 with heparin hexasaccharide using four (4) variations of the stratification scheme described above, based on a combination of SMD and BEUS simulations. The results of the inventive method based on BER disclosed herein are now compared with those obtained from unrestrained BEUS simulations along d as well as FEP, both of which are standard BAE methods. The results are also compared to the RBEUS method.

TABLE 1
Summary of free energy calculation results.

| | MM-GBSA | BEUS | FEP | FEP | RBEUS | BER |
|---|---|---|---|---|---|---|
| Sim. Time (ns) | 0.02 | 0.31 | 0.24 | 1.1 | 1.1 | 0.85 |
| ΔG° | −95 ± 11 | −16.0 ± 1.2 | −14.9 ± 0.7 | −13.6 ± 0.8 | −8.7 ± 0.7 | −8.2 ± 0.6 |
| (kcal/mol) | | | | | | |
| K_{d} (μM)** | O(10⁻⁶³) | O(10⁻⁶) | O(10⁻⁵) | O(10⁻⁴) | 0.5 | 1.1 |

*All error estimates are based on one standard deviation (s.d.).
**K_{d} values are determined directly from mean ΔG° values using Equation 2.
***K_{d} = 1.68 ± 0.03 μM; free energy from experimental ΔG° = −7.92 ± 0.01 kcal/mol.

Based on experimental error analysis, the K_{d} value calculated from the inventive BER method is found to be in the micromolar range with an average value of 1.1 μM (using the mean ΔG° estimate) (Table 1). This is in very good agreement with the K_{d} value obtained from ITC experiments, which is 1.68 μm. The free energy calculated from the experimental K_{d} (−7.92 kcal/mol⁷⁹) is also in good agreement with the computationally calculated binding free energy (Table 1). The quantitative agreement between the computational and experimental binding affinity estimates is a great indicator of the accuracy of the absolute binding free energy calculation method.

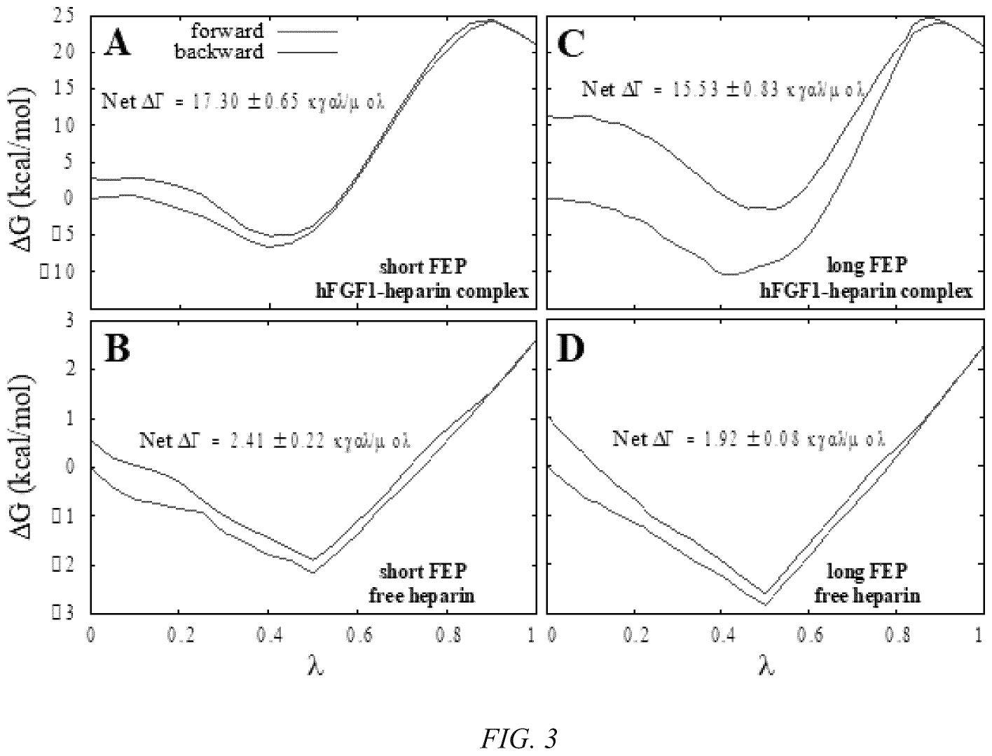

FIGS. 3A and 3B graphically illustrate binding free energy measurements from FEP simulations with the forward and backward transformations shown in red and blue respectively, where free energy change for the double annihilation of heparin hexasaccharide is shown in its bound state (3A) and in its free state (3B)—short FEP, and in its bound state (3C) and in its free state (3D)—long FEP.

Recent computational studies have used the MM-GBSA method to calculate the binding free energy of the hFGF1-heparin interaction, with values ranging from −84.2 kcal/mol to −106.1 kcal/mol (see also Table 1). The results obtained from the MM-GBSA approach are very different from these results, which is to be expected given that methods like MM-PBSA/GBSA typically do not treat entropic or enthalpic contributions to the binding free energy rigorously. On the other hand, these contributors to the binding affinity are typically taken into account during FEP simulations, thus obviating the need for the approximations used in MM-PBSA/GBSA. It is widely accepted that binding free energy estimates from MM-PBSA/GBSA are less accurate than those from FEP, which is considered to be the gold standard for the calculation of absolute binding affinities. Double annihilation FEP was performed to calculate the absolute binding free energy of the hFGF1-heparin complex. The BAR method was used to estimate binding affinities from both the short (60×4 ns) and long (275×4 ns) FEP simulations. An absolute binding free energy of −14.9±0.7 kcal/mol was obtained for the short FEP (FIGS. 3A and 3B), while an absolute binding free energy of −13.61±0.8 kcal/mol was obtained for the long FEP simulations (FIGS. 3C and 3D). Unlike the absolute binding free energy estimated from the BER simulations (−8.7±0.7 kcal/mol) (Table 1), neither of the estimates from the FEP simulations agree with the binding free energy determined from ITC experiments (−7.92 kcal/mol) (Table 1). Hysteresis between the forward and backward directions increases during the long FEP simulations of the hFGF1-heparin complex (FIG. 3C). These results thus show that running longer FEP simulations does not necessarily result in a more accurate binding free energy estimate. The unrestrained BEUS simulation methods also do not provide a reasonable solution to the problem (Table 1). The results of regular unrestrained BEUS simulations are even worse than that obtained from either FEP simulation. The restrained BEUS (RBEUS), on the other hand, improves the BAE considerably getting within 1 kcal/mol of the binding free energy, underestimating the binding affinity only by a factor of 3. However, as previously discussed, the RBEUS method is quite complex in its design and analysis. The BER method is both more accurate and more efficient as compared to the RBEUS method and provides a much more straightforward simulation setup and analysis to estimate the binding free energy and affinity.

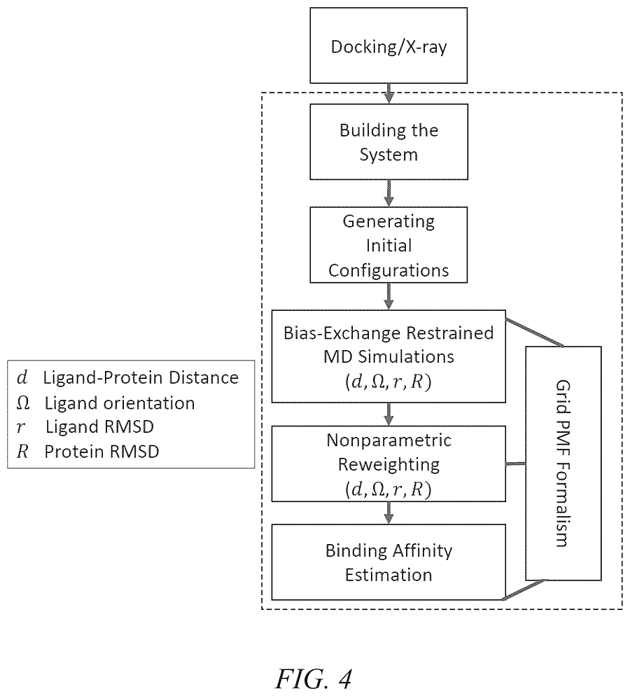

As depicted in FIG. 4, the inventive BER method in one embodiment includes the steps of generating a model by docking or x-ray crystallography, building a simulation therefrom, generating initial configurations representing the ligand and the target molecule, performing BER simulations with four different collective variables (d, Ω, r, and R) as shown in FIG. 5, and using non-parametric reweighting to estimate binding affinity. The outcome of the simulation is significantly influenced by factors such as model quality and the precision of docking.

Studies have shown that the binding affinity and free energy results derived from computational methods can be compared to experimental binding affinities obtained from ITC experiments. However, for a reliable computational free energy estimate, employing purely physics-based free energy calculation methods such as those employed here has proven to be difficult. These examples demonstrate that using the enhanced sampling techniques disclosed herein results in good quantitative agreement between the computational and experimental binding affinity estimates.

The system, method, and computer program for physics-based binding affinity estimation may be implemented in a computer system using hardware, software, firmware, tangible computer-readable media having instructions stored thereon, or a combination thereof, and may be implemented in one or more computer systems or other processing systems.

If programmable logic is used, such logic may execute on a commercially available processing platform or a special purpose device. One of ordinary skill in the art may appreciate that embodiments of the disclosed subject matter can be practiced with various computer system configurations, including multi-core multi-processor systems, minicomputers, mainframe computers, computers linked or clustered with distributed functions, as well as pervasive or miniature computers that may be embedded into virtually any device.

For instance, at least one processor device and a memory may be used to implement the above-described embodiments. A processor device may be a single processor, a plurality of processors, or combinations thereof. Processor devices may have one or more processor “cores.”

Various embodiments of the inventions may be implemented in terms of this example computer system. After reading this description, it will become apparent to a person skilled in the relevant art how to implement one or more of the inventions using other computer systems and/or computer architectures. Although operations may be described as a sequential process, some of the operations may be performed in parallel, concurrently, and/or in a distributed environment and with program code stored locally or remotely for access by single or multi-processor machines. In addition, in some embodiments, the order of operations may be rearranged without departing from the spirit of the disclosed subject matter.

The processor device may be a special purpose or a general-purpose processor device or maybe a cloud service wherein the processor device may reside in the cloud. As will be appreciated by persons skilled in the relevant art, the processor device may also be a single processor in a multi-core/multi-processor system, such system operating alone or in a cluster of computing devices operating in a cluster or server farm. The processor device is connected to a communication infrastructure, for example, a bus, message queue, network, or multi-core message-passing scheme.

The computer system also includes a main memory, for example, random access memory (RAM), and may also include a secondary memory. The secondary memory may include, for example, a hard disk drive or a removable storage drive. The removable storage drive may include a floppy disk drive, a magnetic tape drive, an optical disk drive, a flash memory, a Universal Serial Bus (USB) drive, or the like. The removable storage drive reads from and/or writes to a removable storage unit in a well-known manner. The removable storage unit may include a floppy disk, magnetic tape, optical disk, etc., which is read by and written to by the removable storage drive. As will be appreciated by persons skilled in the relevant art, the removable storage unit includes a computer usable storage medium having stored therein computer software and/or data.

The computer system (optionally) includes a display interface (which can include input and output devices such as keyboards, mice, etc.) that forwards graphics, text, and other data from communication infrastructure (or from a frame buffer not shown) for display on a display unit.

In alternative implementations, the secondary memory may include other similar means for allowing computer programs or other instructions to be loaded into the computer system. Such means may include, for example, the removable storage unit and an interface. Examples of such means may include a program cartridge and cartridge interface (such as that found in video game devices), a removable memory chip (such as an EPROM, PROM, or Flash memory) and associated socket, and other removable storage units and interfaces which allow software and data to be transferred from the removable storage unit to computer system.

The computer system may also include a communication interface. The communication interface allows software and data to be transferred between the computer system and external devices. The communication interface may include a modem, a network interface (such as an Ethernet card), a communication port, a PCMCIA slot, and card, or the like. Software and data transferred via the communication interface may be in the form of signals, which may be electronic, electromagnetic, optical, or other signals capable of being received by the communication interface. These signals may be provided to the communication interface via a communication path. Communication path carries signals, such as over a network in a distributed computing environment, for example, an intranet or the Internet, and may be implemented using wire or cable, fiber optics, a phone line, a cellular phone link, an RF link, or other communication channels.

In this document, the terms “computer program medium” and “computer usable medium” are used to generally refer to media such as removable storage unit, removable storage unit, and a hard disk installed in the hard disk drive. The computer program medium and computer usable medium may also refer to memories, such as main memory and secondary memory, which may be memory semiconductors (e.g., DRAMs, etc.) or cloud computing.

Computer programs (also called computer control logic) are stored in the main memory and/or the secondary memory. The computer programs may also be received via the communication interface. Such computer programs, when executed, enable the computer system to implement the embodiments as discussed herein, including but not limited to machine learning and advanced artificial intelligence. In particular, the computer programs, when executed, enable the processor device to implement the processes of the embodiments discussed here. Accordingly, such computer programs represent controllers of the computer system. Where the embodiments are implemented using software, the software may be stored in a computer program product and loaded into the computer system using the removable storage drive, the interface, the hard disk drive, or the communication interface.

Moreover, embodiments of the disclosure may be practiced with other computer system configurations, including hand-held devices, multi-processor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Embodiments of the inventions also may be directed to computer program products comprising software stored on any computer useable medium. Such software, when executed in one or more data processing devices, causes a data processing device(s) to operate as described herein. Embodiments of the inventions may employ any computer-useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The benefits and advantages described above may relate to one embodiment or may relate to several embodiments. The embodiments are not limited to those that solve any or all of the stated problems or those that have any or all of the stated benefits and advantages. The operations of the methods described herein may be carried out in any suitable order or simultaneously where appropriate. Additionally, individual blocks may be added or deleted from any of the methods without departing from the spirit and scope of the subject matter described herein. Aspects of any of the examples described above may be combined with aspects of any of the other examples described to form further examples without losing the effect sought.

The above description is given by way of example only, and various modifications may be made by those skilled in the art. The above specification, examples, and data provide a complete description of the structure and use of exemplary embodiments. Although various embodiments have been described above with a certain degree of particularity or with reference to one or more individual embodiments, those skilled in the art could make numerous alterations to the disclosed embodiments without departing from the spirit or scope of this specification.

Benefits, other advantages, and solutions to problems have been described above with regard to specific embodiments. However, the benefits, advantages, solutions to problems, and any element(s) that may cause any benefit, advantage, or solution to occur or become more pronounced are not to be construed as critical, required, or essential features or elements of any or all the claims. As used herein, the terms “comprises,” “comprising,” or any other variations thereof are intended to cover a non-exclusive inclusion, such that a process, method, article, or apparatus that comprises a list of elements does not include only those elements but may include other elements not expressly listed or inherent to such process, method, article, or apparatus. Further, no element described herein is required for the practice of the invention unless expressly described as “essential” or “critical.”

The preceding detailed description of exemplary embodiments of the invention makes reference to the accompanying drawings, which show the exemplary embodiment by way of illustration. While these exemplary embodiments are described in sufficient detail to enable those skilled in the art to practice the invention, it should be understood that other embodiments may be realized and that logical and mechanical changes may be made without departing from the spirit and scope of the invention. For example, the steps recited in any of the method or process claims may be executed in any order and are not limited to the order presented. Thus, the preceding detailed description is presented for purposes of illustration only and not of limitation, and the scope of the invention is defined by the preceding description and with respect to the attached claims.

What is claimed is:

1. A system, comprising:
a computer having a processor and a memory; and
a software module stored in the memory, comprising executable instructions that when executed by the processor cause the processor to:
generate a model for a ligand bound to a target molecule; and
estimate a binding affinity between said ligand and said target molecule by:
creating a unified simulation comprising a plurality of replicas, wherein each replica is associated with four restraints along four variables;
exchanging data for said four variables between a subset of said replicas; and
performing a single non-parametric reweighting analysis with said data to estimate an absolute binding free energy for said ligand and said target molecule.

2. The system of claim 1, wherein said processor is configured to generate said model using at least one docking method.

3. The system of claim 1, wherein said processor is configured to generate said model using x-ray crystallography.

4. The system of claim 1, wherein said processor is configured to generate said model by simulating said ligand and said target molecule in a box of water and ions.

5. The system of claim 1, wherein said processor is configured to generate said model by simulating a harmonic restraint on said ligand and said target molecule.

6. The system of claim 1, wherein said four variables comprise a distance between the mass centers of said ligand and said target molecule (d), an orientation of said ligand (Q), a root-mean-square-deviation of said ligand with respect to a reference structure (r_{L}), and a root-mean-square-deviation of said target molecule with respect to said reference structure (rₚ).

7. The system of claim 1, wherein said ligand is a drug, and wherein said target molecule is a protein.

8. A method for physics-based binding affinity estimation, the method comprising the steps of:
creating a unified simulation for a ligand and a target molecule, said unified simulation comprising a plurality of replicas, wherein each replica is associated with four restraints along four variables;
exchanging data for said four variables between a subset of said replicas; and
performing a single non-parametric reweighting analysis with said data to estimate an absolute binding free energy for said ligand and said target molecule.

9. The method of claim 8, wherein said four variables comprise a distance between the mass centers of said ligand and said target molecule (d), an orientation of said ligand (Q), a root-mean-square-deviation of said ligand with respect to a reference structure (r_{L}), and a root-mean-square-deviation of said target molecule with respect to said reference structure (rₚ).

10. The method of claim 8, wherein the step of creating said unified simulation further comprises the step of simulating varying distances between said ligand and said target molecule,
wherein the distance between said ligand and said target molecule in a first replica reflects said ligand and said target molecule when completely bound,
wherein the distance between said ligand and said target molecule in a second replica reflects said ligand and said target molecule when completely unbound, and
wherein the distances between said ligand and said target molecule in the remaining replicas falls between those of the first replica and the second replica.

11. The method of claim 10, wherein step of simulating varying distances between said ligand and said target molecule further comprises performing all the simulations simultaneously.

12. The method of claim 8, wherein the step of exchanging data for said four variables further comprises the steps of:
establishing exchange rules to connect said plurality of replicas, wherein each replica is associated with one of a plurality of nodes in the unified simulation; and
using said replicas to move between said nodes.

13. The method of claim 8 further wherein said replicas have different centers for said four restraints.

14. The method of claim 8, wherein the step of performing said single non-parametric reweighting analysis further comprises:
estimating a likelihood of finding said ligand in the bulk versus finding said ligand at a specific orientation and conformation within a binding pocket of the target molecule;
estimating a difference between flexibility for said ligand in the bulk versus within said binding pocket in terms of movement;
estimating a difference between flexibility for said ligand in the bulk versus within said binding pocket in terms of fluctuations in root-mean-square deviation; and
estimating a difference between flexibility for said ligand in the bulk versus within said binding pocket in terms of orientational changes.

15. The method of claim 8, wherein the step of performing said single non-parametric reweighting analysis further comprises the step of estimating AG° according to the equation
$$Δ {G}^{0} = - RT ln \frac{{\sum }_{i} {w}_{i} {𝒳}_{i}^{pocket}}{{\sum }_{i} {w}_{i} {𝒳}_{i}^{bulk} {p}_{i}} .$$

16. The method of claim 8, further comprising the step of recording said data exchanged between said subset of replicas.

17. A method for physics-based binding affinity estimation, the method comprising the steps of:
generating a model for a ligand bound to a target molecule;
creating a unified simulation for said ligand and said target molecule, said unified simulation comprising a plurality of replicas, wherein each replica is associated with four restraints along four variables;
exchanging data for said four variables between a subset of said replicas;
recording said data; and
performing a single non-parametric reweighting analysis with said data to estimate an absolute binding free energy for said ligand and said target molecule.

18. The method of claim 17, wherein said four variables comprise a distance between the mass centers of said ligand and said target molecule (d), an orientation of said ligand (Q), a root-mean-square-deviation of said ligand with respect to a reference structure (r_{L}), and a root-mean-square-deviation of said target molecule with respect to said reference structure (rp).

19. The method of claim 17, wherein the step of exchanging data for said four variables further comprises the steps of:
establishing exchange rules to connect said plurality of replicas, wherein each replica is associated with one of a plurality of nodes in the unified simulation; and
using said replicas to move between said nodes.

20. The method of claim 17, wherein the step of performing said single non-parametric reweighting analysis further comprises:
estimating a likelihood of finding said ligand in the bulk versus finding said ligand at a specific orientation and conformation within a binding pocket of the target molecule;
estimating a difference between flexibility for said ligand in the bulk versus in said binding pocket in terms of movement;
estimating a difference between flexibility for said ligand in the bulk versus in said binding pocket in terms of fluctuations in root-mean-square deviation for said ligand; and
estimating a difference between flexibility for said ligand in the bulk versus in said binding pocket in terms of orientational changes.
